(19) United States
(12) Patent Application Publication
(10) Pub. No.: US 2023/0225571 A1
(43) Pub. Date: Jul. 20, 2023

(54) PORTABLE, SELF-ADHESIVE DISINFECTANT WIPE CONTAINER AND METHOD THEREFOR

(71) Applicant: JOSE ROBERT T. BATUNGBACAL, IV, LAS VEGAS, NV (US)

(72) Inventor: JOSE ROBERT T. BATUNGBACAL, IV, LAS VEGAS, NV (US)

(21) Appl. No.: 18/095,770

(22) Filed: Jan. 11, 2023

Related U.S. Application Data
(60) Provisional application No. 63/300,317, filed on Jan. 18, 2022.

(51) Int. Cl. A47K 10/42; A61L 2/26; A61L 2/18

(52) U.S. Cl. CPC A47K 10/421; A61L 2/26; A61L 2/18; A47K 2010/3266

(57) ABSTRACT

A portable disinfectant wipe system has a dispenser for holding disinfectant wipes. A plurality of disinfectant wipes is contained within the dispenser. An adhesive material is attached to a rear surface of the dispenser.

TECHNICAL FIELD

The present application generally relates to wet wipes and, more particularly, to a container for storing antiseptic wipes which may be attached to a wall or other surface and then removed and attached to another wall/surface.

### BACKGROUND

Wet wipes and related products have been used for some time by consumers for various cleaning and wiping tasks. These wet wipes are generally pre-wetted with chemicals for various cleaning applications, Most wet-wipes may pre-packaged in a disposable container. These containers generally have a cap/lid through which the wipes may be drawn successively as required. The problem with most of these disposable containers are that they are often misplaced and difficult to locate when needed. For example, many individuals may place the disposable container inside a cabinet, in a desk drawer or similar places in order to organize and clean-up room. Unfortunately, someone who enters the room may not know where the previous party has placed the disposable container and may have to spend a lot of time looking for the wet-wipes. This may be an inefficient use of time especially in time sensitive areas such as the medical field, educational field and like similar areas.

Presently, there are different types of wall mounted wet-wipe dispensers. Many of these types of dispensers are large plastic dispensers similar to paper towel dispensers. Unfortunately, all wall mounted wet-wipe dispensers do not provide the mobility of portable wipe containers. Further, most wall mounted wet-wipe dispensers are bulky and unsightly.

Therefore, it would be desirable to provide a system and method that overcomes the above.

### SUMMARY

In accordance with one embodiment, a portable disinfectant wipe system is disclosed. The portable disinfectant wipe system has a dispenser for holding disinfectant wipes. A plurality of disinfectant wipes is contained within the dispenser. An adhesive material is attached to a rear surface of the dispenser.

In accordance with one embodiment, a portable disinfectant wipe system is disclosed. The portable disinfectant wipe system has a dispenser for holding disinfectant wipes. The dispenser has a. body member formed of a leak resistant material. A perforated opening is formed in a top surface of the body member. A resealing strip is coupled adjacent to the perforated opening. A cover assembly is attached to the top surface of the body member and enclosing the perforated opening. A plurality of disinfectant wipes is contained within the dispenser. An adhesive material is attached to a rear surface of the dispenser, wherein the adhesive material is double side tape.

In accordance with one embodiment, a portable disinfectant wipe system is disclosed. The portable disinfectant wipe system has a dispenser for holding disinfectant wipes. The dispenser has a. body member formed of a leak resistant material. A perforated opening is formed in a top surface of the body member. A resealing strip is coupled adjacent to the perforated opening. A cover assembly is attached to the top surface of the body member and encloses the perforated opening to form an airtight seal. The cover assembly has a frame attached to the top surface of the body member and surrounding the perforated opening. A cover is hingly attached to the frame. A locking tab is formed on the cover securing the cover to the frame. A plurality of disinfectant wipes is contained within the dispenser. A reusable adhesive material is attached to a rear surface of the dispenser. The adhesive material is a plurality of strips of double side tape.

### BRIEF DESCRIPTION OF THE DRAWINGS

The present application is further detailed with respect to the following drawings. These figures are not intended to limit the scope of the present application but rather illustrate certain attributes thereof. The same reference numbers will be used throughout the drawings to refer to the same or like parts.

FIG. 1 is an elevated rear prospective view of an exemplary embodiment of a self-adhesive antiseptic wipe container in accordance with an embodiment of the present invention;

FIG. 2 is an elevated top prospective view of an exemplary embodiment of the self-adhesive antiseptic wipe container with the top cover closed in accordance with an embodiment of the present invention; and

FIG. 3 is an elevated top prospective view of an exemplary embodiment of the self-adhesive antiseptic wipe container with the top cover open and a wet-wipe exposed in accordance with an embodiment of the present invention.

### DESCRIPTION OF THE APPLCIATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary system and method disclose a self-adhesive antiseptic wipe dispenser and method. The self-adhesive antiseptic wipe dispenser may be easily attached and removed from a wall/surface providing the flexibility of disposable wet-wipe containers but the visibility and convenience of a wall mounted wet-wipe dispenser.

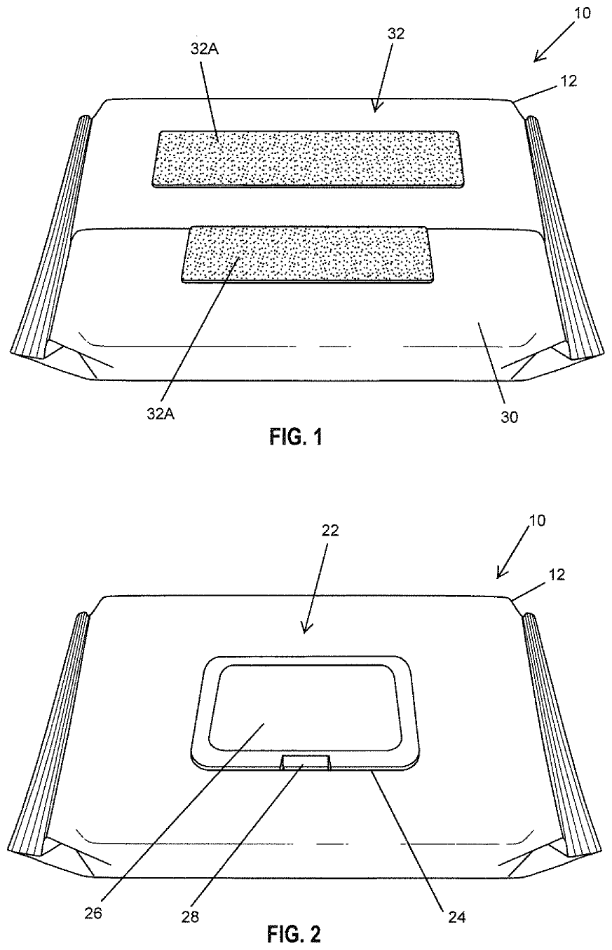

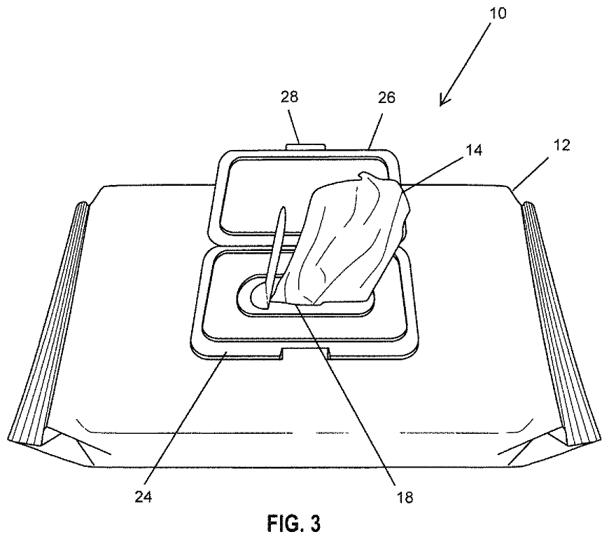

Referring to the FIGS. 1-3, one embodiment of a self-adhesive antiseptic wipe container 10 (hereinafter container 10) may be seen. The container 10 may include a body member 12. The body member 12 may be an enclosed pouch or similar item. The body member 12 may be formed of a leak resistant material in order to contain the liquid components used in the antiseptic wipes 14 prepacked within the container 10. The teak resistant material for the body member 12 may form a complete package for the antiseptic wipes 14 contained within the body member 12.

In accordance with one embodiment, the body member 12 may be formed from a flexible plastic-based material such PET (polyester)+PE (Poly Ethylene), PET (polyester)+METPET (Metallized polyester*)+PE (Poly Ethylene), Paper +ALU (Aluminum)+PE (Poly Ethylene) and similar compounds. The above listing is given as examples and should not be seen in a limiting manner. The container 10 may be formed of other materials without departing from the spirit and scope of the present application.

On an upper surface 16 of the body member 12, a perforated opening 18 may be formed. Pressing on the perforated opening 18 may expose the wet-wipes 14 prepacked within the container 10 allowing one to remove the uppermost antiseptic wipe 14. A resealing strip 20 may be coupled and adjacent to the perforated opening 18. The resealing strip 20 may be used to reseal the perforated opening 18 to prevent the antiseptic wipes 14 from drying out.

A cover assembly 22 may be attached to the top surface 16 of the body member 12. The cover 22 may be used to enclose the perforated opening 18. The cover assembly 22 may be formed of a different material from the body member 12, In accordance with one embodiment, the cover assembly 22 may be formed of a hard plastic suitable for repeated use in consumer product packaging. In accordance with one embodiment, the cover assembly 22 may include a frame 24. The frame 24 may be directly attached to the top surface 16 of the body member 12. A cover member 26 may be hingely attached to the frame 24 and may be raised and lowered from the frame 24 to expose and cover the perforated opening 18. The cover member 26 and the frame 24 may form a substantially airtight seal when closed to prevent the antiseptic wipes 14 from drying out. A locking tab 28 may be formed on the cover member 26. The locking tab 28 may be used to secure the cover member 26 to the frame

On a rear surface 30 of the body member 12 is a reusable adhesive 32. The reusable adhesive 32 may be used to attach the container 10 to a wall or other surface (hereinafter wall) in a room. After the device 10 has been attached to the wall, a user may pull on the device 10 to remove the device 10 from the current wall and then place the device 10 on another wall or another area of the present wall. In accordance with one embodiment, the reusable adhesive 32 may be double side adhesive tape 32A. The double side adhesive tape 32A should be strong enough to secure the device 10 to the wall but not so strong as to damage the wall when removing the device 10 from the wall. For example, the double side adhesive tape 32A may be 5-to-15-pound double side adhesive tape 32A. However, other weighted double side adhesive tape 32A may be used as long as the double side adhesive tape 32A does not damage the wall when removing the device 10 from the wall.

In the present embodiment, two strips of double side adhesive tape 32A may be seen. However, this is only shown as an example and any number of strips double side adhesive tape 32A may be used.

The foregoing description is illustrative of particular embodiments of the application, but is not meant to be a limitation upon the practice thereof. The following claims, including all equivalents thereof, are intended to define the scope of the application.

What is claimed is:

1. A portable disinfectant wipe system comprising:
a dispenser for holding disinfectant wipes;
a plurality of disinfectant wipes contained within the dispenser; and
an adhesive material attached to a rear surface of the dispenser.

2. The portable disinfectant wipe system of claim 1, wherein the dispenser comprises:
a body member formed of a leak resistant material;
a perforated opening formed in a top surface of the body member;
a resealing strip coupled adjacent to the perforated opening; and
a cover assembly attached to the top surface of the body member and enclosing the perforated opening.

3. The portable disinfectant wipe system of claim 2, wherein the cover assembly comprises:
a frame attached to the top surface of the body member and surrounding the perforated opening; and
a cover hingly attached to the frame.

4. The portable disinfectant wipe system of claim 3, wherein the cover assembly comprises a locking tab formed on the cover securing the cover to the frame.

2. portable disinfectant wipe system of claim 2, wherein the cover assembly forms an airtight seal.

6. The portable disinfectant wipe system of claim 1, wherein the adhesive material is a reusable adhesive allowing the system to be removable attached to a surface,

7. The portable disinfectant wipe system of claim 1, wherein the adhesive material is a double side adhesive tape allowing the system to be removable attached to a surface.

8. The portable disinfectant wipe system of claim 1, wherein the adhesive material is a plurality of double side adhesive tapes allowing the system to be removable attached to a surface.

9. The portable disinfectant wipe system of claim 1, wherein the adhesive material is a double side adhesive tape having an adhesive strength of at least 5 pounds.

10. A portable disinfectant wipe system comprising:
a dispenser for holding disinfectant wipes, wherein the dispenser comprises:
a body member formed of a leak resistant material;
a perforated opening formed in a top surface of the body member;
a resealing strip coupled adjacent to the perforated opening; and
a cover assembly attached to the top surface of the body member and enclosing the perforated opening;
a plurality of disinfectant wipes contained within the dispenser; and.
an adhesive material attached to a rear surface of the dispenser, wherein the adhesive material is double side tape.

11. The portable disinfectant wipe system of claim 10, wherein the cover assembly comprises:
a frame attached to the top surface of the body member and surrounding the perforated opening; and
a cover hingly attached to the frame.

12. The portable disinfectant wipe system of claim 11, wherein the cover assembly comprises a locking tab formed on the cover securing the cover to the frame.

13. The portable disinfectant wipe system of claim 10, wherein the cover assembly forms an airtight seal.

14. The portable disinfectant wipe system of claim 10, wherein the adhesive material is a reusable adhesive allowing the system to he removable attached to a surface.

1. 5. The portable disinfectant wipe system of claim 10. wherein the adhesive material is a double side adhesive tape allowing the system to be removable attached to a surface.

16. The portable disinfectant wipe system of claim 10, wherein the adhesive material is a plurality of double side adhesive tapes allowing the system to be removable attached to a surface.

17. portable disinfectant wipe system of claim 10, wherein the adhesive material is a double side adhesive tape having an adhesive strength of at least 5 pounds.

18. A portable disinfectant wipe system comprising:
a dispenser for holding disinfectant wipes, wherein the dispenser comprises:
a body member formed of a leak resistant material;
a perforated opening formed in a top surface of the body member;
a resealing strip coupled adjacent to the perforated opening; and
a cover assembly attached to the top surface of the body member and enclosing the perforated opening and forming an airtight seal, wherein the cover assembly comprises:
a frame attached to the top surface of the body member and surrounding the perforated opening;
a cover hingly attached to the frame; and
a locking tab formed on the cover securing the cover to the frame.
a plurality of disinfectant wipes contained within the dispenser; and
a reusable adhesive material attached to a rear surface of the dispenser, wherein the adhesive material is a plurality of strips of double side tape.

19. The portable disinfectant wipe system of claim 18, wherein the adhesive material is a double side adhesive tape having an adhesive strength of at least 5 pounds.
